(19) United States
(12) Patent Application Publication
(10) Pub. No.: US 2017/0002389 A1
(43) Pub. Date: Jan. 5, 2017

(54) METHOD OF PREPARING SEAWEED-DERIVED GALACTOSE USING AGARASE

(71) Applicant: Hyundai Motor Company, Seoul (KR)

(72) Inventors: Chae Hwan Hong, Seoul (KR); Na Kyong Yun, Gyeonggi-do (KR); SungWan Jeon, Gyeonggi-do (KR)

(21) Appl. No.: 14/876,083

(22) Filed: Oct. 6, 2015

(30) Foreign Application Priority Data
Jul. 1, 2015 (KR) 10-2015-0094302

(51) Int. Cl. C12P 19/02; C13K 13/00

(52) U.S. Cl. CPC C12P 19/02; C13K 13/007; C12P 2201/00

(57) ABSTRACT

Provided is a method of preparing galactose by enzymatic treatment of a red algae residue. The method of preparing galactose comprises: preparing a residue; treating the residue with an enzyme; concentrating the enzyme treated residue containing sugar mixture, and precipitating and particulating galactose by adding an alcohol to the concentrated sugar mixture. The preparation method according to the present invention provides a technique capable of industrially producing a substantial amount of galactose which is utilized as an important intermediate material in the preparation of biochemical materials.

### CROSS-REFERENCE TO RELATED APPLICATION

This application claims the benefit of priority Korean Patent Application No. 10-2015-0094302 filed on Jul. 1, 2015 with the Korean Intellectual Property Office, the entire contents of which are incorporated herein by reference.

### TECHNICAL FIELD

The present invention relates to a method of preparing galactose by enzymatic treatment of seaweed such as red algae residue. Particularly, the method of preparing galactose comprises: preparing a seaweed residue, treating the seaweed residue with an enzyme, and concentrating, precipitating and particulating galactose contained in the seaweed residue.

### BACKGROUND

Galactose is one of carbohydrate components in marine algae such as a red algae and can have useful functions when applied in the development of raw materials for chemical reactions and physiologically active materials, and pharmaceutical fields.

Galactose is an aldohexose that is rarely seen in a free form in nature but widely distributed in a polymer form, and has a molecular formula of C₆H₁₂O₆ and a melting point of about 167° C. Galactose may be prepared in a white powder with a sweet taste which may be easily dissolved in water, and may have a melting point of about 118° C. when water is contained with crystallization. Galactose may be a D- and L-type optical isomer while D-galactose is commonly existing galactose.

Current studies on the preparation of galactose as a bio-sugar have focused on a saccharification technique using acidic materials, but the saccharification technique using acidic materials has not been commercialized yet. Since the saccharification technique using acidic materials requires use of acidic chemicals, there is a disadvantage that neutralization of the acidic chemicals is necessary. Further, a high concentration of acid material has been used to maximize degradation of the cell wall of red algae, which may alter a structure of the monosaccharide bio-sugar produced, leading to an increased production of by-products. Problematically, the saccharification yield is decreased.

In addition, galactose prepared by the saccharification process may be a monosaccharide, and galactose may not be obtained as particles directly from a saccharification liquid, because proteins and other impurities of algae components and acid chemicals may be included in the liquid upon acid saccharification.

However, current studies for the technique of preparing bio-sugars such as galactose from algae by the saccharification process have focused on a method of saccharifying algae using acidic chemicals, neutralizing the acid chemicals, and then preparing fuel materials such as bio-ethanol by a fermentation process. A technique of preparing monosaccharide bio-sugars such as galactose as particles directly from a saccharification liquid has not been completed yet.

In addition, since the saccharification liquid which is obtained by the saccharification technique using acidic chemicals contains bio-sugars, utilization methods thereof have been studied. However, there have been no satisfactory studies on enzymatic saccharification of a solid phase which is produced as residues resulting from saccharification using acidic chemicals. Cellulase needed in the preparation of sugars from land plant sources has been studied from about 20 years ago, and its commercial products have been released by the world's biggest makers such as Denmark—Novozymes. However, commercial production of an algal galactan degradation enzyme, agarase has not been achieved yet, and a technique and a method of chemically preparing agarase have not been developed. Therefore, studies on enzymatic saccharification are still in the very early stages.

Such enzymatic saccharification technique for solid-phase materials remaining after saccharification using acidic chemicals may be industrially valuable, and economical enzyme preparation, saccharification by using the enzyme, and separation and purification techniques are of great industrial importance.

However, there has been no available process of producing galactose using enzymes after saccharification of algae worldwide, and there has been no report about development of a complete process at the laboratory level. The technologies still remain at the level of reporting the sugar components present in a saccharification liquid by analyzing the liquid after saccharification.

### SUMMARY OF THE INVENTION

In order to solve the problems, the present inventors have investigated that solid-phase galactose particles could be prepared from red algae residues using enzymes by combination of particular unit processes, thereby completing the present invention.

In one aspect, the present invention provides a method of preparing galactose. The method may include: preparing a red algae residue by steps comprising saccharification and filtration of red algae; reacting the red algae residue with an agarase-containing solution to obtain a sugar mixture; filtering the sugar mixture; concentrating the filtered sugar mixture; and precipitating galactose by steps comprising adding an alcohol to the concentrated sugar mixture. The red algae may be one or more selected from the group consisting of the genus Chondrus, Eucheuma, Gigartina, Pterocladia, Hypnea, Iridaea, Kappaphycus, Gellidium, and Gracilaria.

When the red algae residue is prepared, the saccharification may be performed at a temperature of about 80 to 150° C. Preferably, the saccharification may be a hydrolysis of the red algae and the hydrolysis may be performed by steps comprising adding an acid at a concentration of about 0.1% (w/v) to 15% (w/v). Particularly, the acid may be one or more selected from the group consisting of sulfuric acid (H₂SO₄), hydrochloric acid (HCl), bromic acid (HBr), nitric acid (HNO₃), acetic acid (CH₃COOH), formic acid (HCOOH), perchloric acid (HClO₄), phosphoric acid (H₃PO₄), and para toluenesulfonic acid (PTSA).

When the red algae residue is prepared, the filtration of red algae may be performed by silica gel chromatography or filtration using a filter.

The agarase may be obtained from Saccharophagus degradans 2-40. Preferably, the agarase-containing solution may be obtained by steps comprising culturing Saccharophagus degradans 2-40; removing Saccharophagus degradans 2-40 from a culture medium; and concentrating an agarase remaining in the culture medium. In particular, Saccharophagus degradans may be cultured at a temperature of about 30 to 40° C. for about 36 to 72 hours.

The sugar mixture may be filtered by steps comprising filtering the sugar mixture using a column chromatography, and additionally filtering the sugar mixture using a microfilter. The column chromatography may contain a silica gel having an average particle diameter of about 0.1 to 0.5 mm and the microfilter may have a pore size of about 0.45 to 0.9 μm. Preferably, the filtering the sugar mixture may be performed at a flow rate of about 0.1 to 100 mL/min.

The filtered sugar mixture may be concentrated by distillation of the filtered sugar mixture under vacuum. Preferably, the concentrating may be performed at a temperature of about 30 to 60° C., at a pressure of about 10 to 120 mbar.

When the galactose is precipitated, the precipitating may be performed at a temperature of about −10° C. to 25° C. Preferably, the alcohol added in the precipitating may be one or more selected from the group consisting of methanol, ethanol, and propanol.

The method of preparing galactose may further include additional filtering after the precipitating to obtain galactose particles.

In another aspect, the present invention provides a galactose prepared by the method as described herein. The galactose thus prepared may include D-galactose, L-galactose, or a mixture thereof. Further, the galactose may be in the form of white solid particles. Preferably, galactose may have a melting point of about 163 to 170° C. and a purity of about 80 wt % or greater.

Other aspects of the present invention are disclosed infra.

### BRIEF DESCRIPTION OF THE DRAWINGS

FIG. 1 is a flow chart showing an exemplary method of preparing galactose according to an exemplary embodiment of the present invention; and

FIG. 2 shows a photograph of exemplary galactose particles obtained by enzymatic treatment according to an exemplary embodiment of the present invention.

### DETAILED DESCRIPTION

The terminology used herein is for the purpose of describing particular exemplary embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Hereinafter, a resin composite and a method for preparing galactose from a red algae or red algae residue according to various exemplary embodiments will be explained in detail.

Polysaccharides constituting the cell walls of red algae include cellulose, xylan, mannan, agar and carrageenan. For example, agar has been known to be a major component of viscous polysaccharides which constitute the outer layer and intercellular spaces of the red algal cell wall.

Agar is a polymer consisting of galactose and 3,6-anhydro-L-galactose (AHG) as a unit, which are alternately bonded by α-1,3-linkage and β-1,4-linkage (Kazlowski B, Pan C L, Ko Y T. (2008). Separation and quantification of neoagaro and agaro-oligosaccharide products generated from agarose digestion by beta agarase and HCl in liquid chromatography systems. Carbohydrate Research, 343, 2443-2450). A unit of 3,6-anhydro-L-galactose and galactose linked via α-1,3-linkage, which is called neoagarbiose, has been analyzed using NMR spectrometry that 3,6-anhydro-L-galactose and galactose (D-glactose) constitute neoagarobiose (Kim H T, Lee S, Lee D et al. (2009), Overexpression and molecular characterization of Aga 50D from saccharophagus degradans 2-40. Applied microbiology and biotechnology, 1432-0614 (Online)).

Since an agar degrading marine microorganism, e.g. Bacillus gelaticus, was first isolated by Gran in 1902, the genus Agarivorans, Alteromonas, Cytophaga, and Microbulbifer have been reported (Swartz M N, Nancy G. (1958). Agarase from an agar-digesting bacterium. Journal of bacteriology, 77, 403-409.).

Among others, Saccharophagus degradans 2-40 has been known as an aerobic rod-shaped, gamma-subgroup proteobacterium capable of degrading many complex polysaccharides such as agarose (Ekborg N A, Gonzalez J M, et al. (2005). Saccharophagus degradans gen. nov., sp. Nov., a versatile marine degrader of complex polysaccharides. International journal of systematics and evolutionary microbiology, 55, 1545-1549.). Formerly, it was classified as the genus Microbulbifer, but was officially named as Saccharophagus degradans 2-40 in 2005. The genome sequence of the strain (U.S. Department of Energy Joint Genome Institute) was revealed in 2008 (Weiner R M, Talyor L E, Henrissat B, et al. (2008). Complete genome sequence of the complex carbohydrate-degrading marine bacterium, Saccharophagus degradans strain 2-40, PLOS Genetics, 4, e1000087).

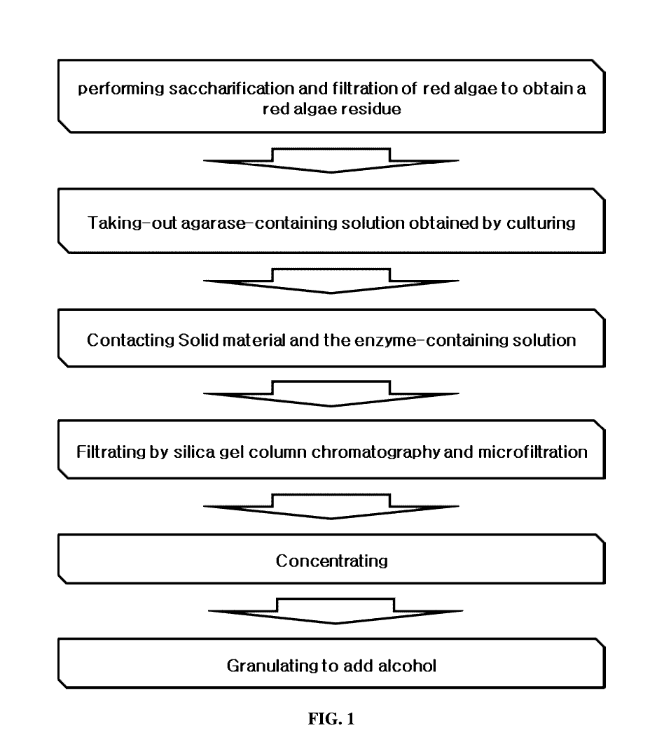

Accordingly, in one aspect, the present invention provides a method of preparing galactose. The method, as shown in FIG. 1, may include: preparing a red algae residue by steps comprising saccharification and filtration of red algae; reacting the red algae residue with an agarase-containing solution to obtain a sugar mixture; filtering the sugar mixture; concentrating the filtered sugar mixture; and precipitating galactose by steps comprising adding alcohol to the concentrated sugar mixture.

In an exemplary embodiment, the saccharification of the red algae may be performed at a temperature of about 80 to 150° C., of about 100 to 150° C., or particularly of about 120 to 150° C., but is not limited thereto.

Further, the saccharification process may be a hydrolysis of the red algae, in which the hydrolysis may be performed by adding an acid at a concentration of about 0.05 to 15% (w/v), of about 0.05 to 10% (w/v), of about 0.05 to 5% (w/v), of about 0.1 to 15% (w/v), of about 0.1 to 10% (w/v), of about 0.1 to 5% (w/v), of about 0.5 to 15% (w/v), of about 0.5 to 10% (w/v), of about 0.5 to 5% (w/v), of about 1 to 15% (w/v), of about 1 to 10% (w/v), or particularly of about 1 to 5% (w/v), but is not limited thereto.

Further, the acid may be one or more selected from the group consisting of sulfuric acid (H₂SO₄), hydrochloric acid (HCl), bromic acid (HBr), nitric acid (HNO₃), acetic acid (CH₃COOH), formic acid (HCOOH), perchloric acid (HClO₄), phosphoric acid (H₃PO₄), and para-toluenesulfonic acid (PTSA), but is not limited thereto.

Further, the red algae may contain or produce galactose or a polymer thereof. Particularly, the red algae may belong to the genus of Chondrus, Eucheuma, Gigartina, Pterocladia, Hypnea, Iridaea, Kappaphycus, Gellidium, or Gracilaria. For example, a red algae such as Gracilaria or Gellidium (agar) may be used, but is not limited thereto. Further, the red algae may be provided as a dry product of the raw red algae, a dry product after washing the raw red algae, or a powder thereof, but is not limited thereto.

Further, the filtration for preparing the red algae residue may be performed at least using column chromatography or using a filter, but is not limited thereto. Any filter generally used in the arts may be used without limitation.

In particular, the column chromatography may contain a silica gel or silica resin. The silica gel may be in a form of particle which may have an average particle size of about 0.1 to 0.5 mm, of about 0.1 to 0.4 mm, of about 0.1 to 0.3 mm, or particularly of about 0.1 to 0.2 mm. The silica gel may be various neutral silica gels may be used, but the silica gel is not limited to particular silica materials.

The filter may have a pore size of about 1 to 20 μm, of about 1 to 15 μm, of about 1 to 10 μm, of about 3 to 20 μm, of about 3 to 15 μm, of about 3 to 10 μm, of about 5 to 20 μm, of about 5 to 15 μm, or particularly of about 5 to 10 μm, but is not limited thereto.

Thus prepared red algae residue may be obtained as a solid remaining after filtration. The red algae residue may subsequently react with an agarase-containing solution to obtain a sugar mixture which may contain galactose produced in red algal cell walls. The sugar mixture may be a liquid, for example, an aqueous solution.

Compared to a saccharification technique using acidic materials and other chemicals, the method of obtaining the sugar mixture containing galactose by enzymatic treatment such as agarase may greatly reduce production costs. For instance, the method using enzymatic treatment does not require a process of neutralizing the acidic materials which are injected during saccharification using acidic materials. In addition, the method may further improve operation stability and efficiency during a concentration process.

The reaction may be performed at a reaction temperature of about 30 to 70° C., of about 30 to 60° C., of about 40 to 70° C., of about 40 to 60° C., or particularly of about 50° C., but is not limited thereto.

Further, the reaction may be performed for a reaction time of about 48 hrs or longer, for example, of about 48 to 96 hrs, of about 48 to 84 hrs, of about 48 to 72 hrs, or particularly of about 48 to 60 hrs, but is not limited thereto.

The agarase may be obtained from a culture containing a microorganism or a strain that produce, by steps comprising culturing the strain capable of producing the agarase, and removing the strain from the culture medium.

The agarase may be obtained or produced from a strain such as Bacillus, Agarivorans, Alteromonas, Cytophaga, Microbulbifer, Saccharophagus, and the like, and for example, the agarase may be produced from Saccharophagus degradans 2-40.

The strain may be cultured at a temperature of about 30 to 40° C., of about 33 to 40° C., of about 35 to 40° C., or particularly of about 37° C., for about 36 to 72 hrs, 36 to 66 hrs, 36 to 60 hrs, 36 to 52 hrs, 42 to 72 hrs, 42 to 66 hrs, 42 to 60 hrs, 42 to 52 hrs, or 48 hrs, to suitably or substantially produce the ararase, but the culturing condition may not be limited thereto.

Further, a culturing medium for producing agarase in the above described strain may be prepared by adding about 2.3% artificial seawater, about 0.5% ammonium chloride, 1.5% agar, and 50 mM Tris-HCl to 1 L of water.

The artificial seawater may be prepared to have a chemical composition identical to seawater of 35‰ salinity. For example, the artificial seawater may be prepared by mixing Solution 1 and Solution 2, in which Solution 1 is prepared by dissolving about 23.9 g of NaCl, about 4.0 g of Na₂SO₄, about 0.7 g of NaHCO₃, about 0.1 g of KBr, about 30 mg of H₃BO₃ and about 3 mg of NaF in 500 ml of distilled water, and Solution 2 is prepared by dissolving about 10.8 g of MgCl₂.6H₂O, about 1.5 g of CaCl.2H₂O, and about 25 mg of SrCl₂.6H₂O in 455 ml of distilled water.

Filtering may include a primary filtration using a silica gel column chromatography, and a secondary filtration using a microfilter after the primary filtration step.

The primary filtration may be performed one or more cycles, for example, 1 to 10 cycles, 1 to 5 cycles, or 1 to 3 cycles using silica gel column chromatography. Particularly, the silica gel column chromatography may remove impurities, such as any particles and proteins components from the sugar mixture.

Further, the silica gel particle may have an average particle size of about 0.1 to 0.5 mm, of about 0.1 to 0.4 mm, of about 0.1 to 0.3 mm, or of about 0.1 to 0.2 mm, and various neutral silica gels may be used, but the silica gel is not limited to particular silica materials. Further, the volume of silica gel packed in the silica gel column may be suitably ½ to ⅕ with respect to the volume of the sugar mixture to be injected. If the volume ratio exceeds the range, separation efficiency may be reduced or the content of impurity particles may be increased to generate a problem that silica gel particles may coexist with galactose particles in a final particulation process.

Further, the microfilter used in the secondary filtration may have a pore size of about 0.45 to 0.9 μm. When a filter having a pore size of 0.45 μm or less is used, operation stability of the filtration process may be reduced, resulting in an uneconomic process. When a filter having a pore size of greater than 0.9 μm is used, some microparticles may not be filtered to reduce purity of the final product.

A flow rate of the sugar mixture in the column chromatography may be of about 0.1 to 100 mL/min, of about 0.1 to about 80 mL/min, of about 0.1 to about 60 mL/min, of about 0.1 to about 40 mL/min, of about 0.1 to 20 mL/min, of about 0.1 to 10 mL/min, of about 0.1 to 5 mL/min or particularly of about 3.5 mL/min, but is not limited thereto. A flow rate suitable for the large-scale system may be suitably adjusted.

The filtered sugar mixture, for example, by the primary and the secondary filtration, may be concentrated. For instance, the sugar mixture may be concentrated by distillation under vacuum to remove water or other solvent, using a vacuum distillation unit, but is not limited to particular devices. Preferably, the sugar mixture may be concentrated to a volume of about 1/10 to 1/20 of the volume of the filtered sugar mixture.

Further, the concentrating the sugar mixture may be performed at a temperature of about 30 to 60° C. When the temperature is greater than about 60° C., partial discoloration of galactose to be concentrated may occur. When the temperature is less than 30° C., the processing time may be increased.

Further, the concentrating may be performed at a pressure of about 10 to 120 mbar. When the pressure is greater than 120 mbar, the processing time may be increased. When the pressure is 10 mbar or less, the process stability may be reduced.

From the concentrated sugar mixture, galactose may be precipitated. Particularly, alcohol may be added to the concentrated sugar mixture to induce formation of galactose particles (particulation), thereby precipitating the galactose. The precipitation may be performed at a temperature of about −10° C. to 25° C. or alternatively, alcohol at a temperature of about −10° C. to 25° C. may be added to the concentrated sugar mixture. The precipitated galactose solid particles may be obtained using a vacuum filtering device.

The alcohol may be one or more selected from the group consisting of linear or branched alcohols having 1 to 4 carbon atoms, for example, methanol, ethanol, and propanol (e.g., isopropyl alcohol), but is not limited thereto.

The concentration of alcohol may be of about 10 to 100% (v/v), of about 20 to 100(v/v), 30 to 100(v/v), of about 40 to 100(v/v), of about 50 to 100(v/v), of about 60 to 100(v/v), of about 70 to 100(v/v), of about 80 to 100(v/v), of about 90 to 100(v/v), of about 95 to 100(v/v), or of about 98 to 100(v/v), for example, about 99% (v/v). In a particular embodiment, the alcohol may be alcohol having a concentration of about 10 to 100% (v/v), of about 20 to 100(v/v), of about 30 to 100(v/v), of about 40 to 100(v/v), of about 50 to 100(v/v), of about 60 to 100(v/v), of about 70 to 100(v/v), of about 80 to 100(v/v), of about 90 to 100(v/v), of about 95 to 100(v/v), or of about 98 to 100(v/v), for example, 99% (v/v) methanol, ethanol, and propanol (e.g., isopropyl alcohol).

The injection volume of the alcohol may be suitably of about 5 to 10 times the volume of the concentrated sugar mixture. When the volume is about 5 times or less, precipitation of particles may not properly occur. When the volume is about 10 times or greater, costs may be increased by excessive use thereof.

The galactose prepared by the method of preparing galactose may include D-galactose, L-galactose, or a mixture thereof. Further, the galactose may be obtained in the form of white solid particles or powder. If galactose is obtained in the form of solid particles, there are advantages of easy weighing, precise control of injection amount, easy storage, and reduction in volume, when galactose is used as a starting material of other chemical reactions. If liquid-phase galactose is used as a starting material of subsequent chemical reactions, the above described advantages may not be expected, and thus the production process may become very difficult.

Another aspect provides galactose prepared by the method of preparing galactose.

The galactose prepared by the method of preparing galactose may include D-galactose, L-galactose, or a mixture thereof. Further, the galactose may be in the form of white solid particles, but is not limited thereto.

Further, the galactose may have a melting point of about 163 to 170° C., for example, of about 167 to 169° C. Considering that pure galactose has a melting point of 167 to 169° C., the galactose may be galactose having a purity of about 80 wt % or greater, 85 wt % or greater, 90 wt % or greater, 95 wt % or greater, 96 wt % or greater, 97 wt % or greater, 98 wt % or greater, or 99 wt % or greater.

The present invention relates to a method of preparing galactose by enzymatic treatment of red algae residue. Particularly, the method of preparing galactose may include preparing a residue, reacting the residue with an enzyme, concentrating a produced sugar mixture, and precipitating and particulating galactose contained in the sugar mixture.

The preparation method according to the present invention suggests an industrially important technique of obtaining galactose with a high yield, for example, not by discarding various solid materials obtained from the conventional saccharification process using acidic chemicals but by treating them with enzymes. In addition, this method provides economic advantages for fishing villages cultivating red algae, and solves the environmental problems caused by leaving algae unattended.

Hereinafter, the present invention will be described in more detail with reference to the following Examples. However, these Examples are for illustrative purposes only, and the scope of the invention is not intended to be limited by these Examples.

### EXAMPLE

### Preparation Example

### Preparation of Agarase-Containing Solution

A marine strain, Saccharophagus degradans 2-10 (available from ATCC (American Type Culture Collection), Manassas, Va., USA) was cultured.

In detail, a medium composition was 2.3% artificial seawater (product name: Aquarium Systems, Mentor, Ohio), 0.5% ammonium chloride (Sigma-Aldrich Corp.), 50 mM Tris-HCl (Sigma-Aldrich Corp.), and 1.5% agar (Sigma-Aldrich Corp.) in 1 L of water, and the culturing was performed under conditions of a temperature of 35° C. for 48 hours in a 5 L-fermentation reactor (LiFlus GM, Biotron Inc.)

The culture was centrifuged (Continent 512R, Hanil Science Industrial Co.) at 6000 rpm for 30 minutes, thereby obtaining 1000 ml of a liquid supernatant containing Saccharophagus degradans 2-40-derived agarase.

### Example 1

### Preparation of Red Algae-Derived Galactose

Step 1: Preparation of Red Algae Residue

Red algae Gracilaria collected in Chunnam coastal area of South Korea was dried and pulverized. Then, 350 mL of distilled water and 150 mL of 1 N HCl solution were added to a 500 cc-flask, and this solution was adjusted to 0.3 N HCl. 25 g of Gracilaria thus dried and pulverized was added to the solution, followed by agitation at 120° C. for 4 hours. Next, after stopping the agitation, the solution was left at room temperature (25° C.), and filtered using a column packed with silica particles to obtain 15 g of red algae residue.

Step 2: Acquisition of Galactose-Containing Sugar Mixture

10 mL of the agarase-containing solution prepared in Preparation Example was mixed with respect to 1 g of the red algae residue obtained in Step 1. After mixing, the solution was allowed to react at 50° C. for 48 hours to obtain 200 cc of a galactose-containing sugar mixture.

Step 3: Filtration

A primary filtration step was performed by applying the sugar mixture to a column packed with 100 mL of neutral silica gel particles having an average diameter of 0.1 to 0.5 mm at a flow rate of 3.5 mL/min. Next, the liquid materials passed through the silica gel column were subjected to a secondary filtration using a microfilter having an average pore size of 0.45 μm. Through these filtration procedures, 150 ml of liquid sugar mixture, from which solid phase materials were removed, was obtained.

Step 4: Concentration and Particulation (Precipitation)

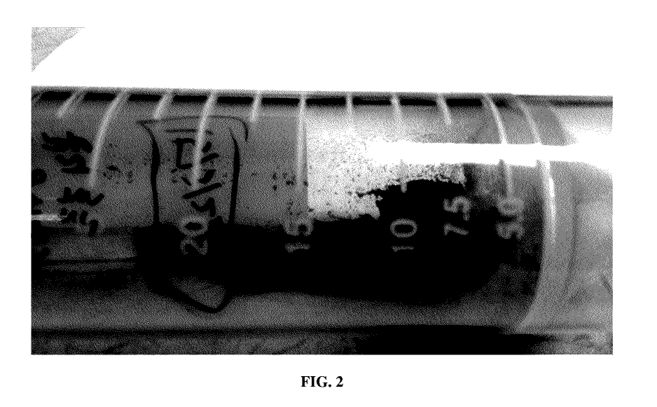

A rotary evaporator (IKA® RV 10) was used to concentrate the liquid sugar mixture to about 1/20 of the volume before concentration at a bath temperature of 55° C. and under reduced pressure of 90 mbar. Then, 99% (v/v) ethanol stored at a low temperature was added to the concentrated material to precipitate particles. In this regard, the volume of the injected ethanol was about 20 times volume of the concentrated material. Thereafter, the precipitated particles were subjected to vacuum filtration to obtain 10 g of galactose particles. The obtained galactose particles are shown in FIG. 2.

### Comparative Example 1

Preparation was performed in the same manner as in Example 1, except that Step 2 was performed using a commercial enzyme, cellulase (Viscozyme; Novozyme) instead of the agarase-containing solution prepared in Preparation Example.

### Comparative Example 2

Preparation was performed in the same manner as in Example 1, except that the concentration and particulation steps were performed without the filtration step.

### Comparative Example 3

Preparation was performed in the same manner as in Example 1, except that the silica gel column chromatography was performed but microfiltration was not performed in the filtration step, and then the concentration and particulation steps were performed.

### Comparative Example 4

Preparation was performed in the same manner as in Example 1, except that 99% (v/v) hexane was used instead of ethanol in the particulation step after concentration. In this regard, the volume of the injected hexane was about 20 times volume of the concentrated material.

### Comparative Example 5

Preparation was performed in the same manner as in Example 1, except that 99% (v/v) ethyl acetate was used instead of ethanol in the particulation step after concentration. In this regard, the volume of the injected ethyl acetate was about 20 times volume of the concentrated material.

### Experimental Example 1

### Test of Galactose Particle Formation

Galactose particle formations of Example 1 and Comparative Examples 1 to 5 were examined. The results are given in the following Table 1.

TABLE 1

| Classification | Particle formation |
|---|---|
| Example 1 | Particles |
| Comparative Example 1 | Sticky materials/No particles |
| Comparative Example 2 | Sticky materials/No particles |
| Comparative Example 3 | Sticky materials/No particles |
| Comparative Example 4 | Sticky materials/No particles |
| Comparative Example 5 | Sticky materials/No particles |

As shown in Table 1, particles were formed in Example 1, whereas sticky materials were only produced and no particles were formed in Comparative Example 1 to 5.

### Experimental Example 2

### Measurement of Melting Point for Test of Galactose Formation

To examine whether galactose was produced in Example 1 and Comparative Examples 1 to 5, differential scanning calorimetry (DSC) (USA, TA instruments) was used to measure their melting point indicating purity thereof.

In detail, the melting point of a galactose reagent was measured as 167 to 169° C. The melting points of Example 1 and Comparative Examples 1 to 5 were measured and the results are given in the following Table 1.

TABLE 2

| | Melting point (° C.) |
|---|---|
| Example 1 | 167 |
| Comparative Example 1 | 65 |
| Comparative Example 2 | 66 |
| Comparative Example 3 | 50 |
| Comparative Example 4 | 45 |
| Comparative Example 5 | 45 |

As shown in Table 2, Example 1 was found to have a melting point of 167° C., which is within the range from 167 to 169° C. of the melting point of the galactose reagent, indicating production of galactose. In contrast, Comparative Examples 1 to 5 were found to have a melting point of 45 to 65° C., which is not within the melting point range of the galactose reagent, indicating no production of galactose.

What is claimed is:

1. A method of preparing galactose, the method comprising
preparing a red algae residue by steps comprising saccharification and filtration of red algae;
reacting the red algae residue with an agarase-containing solution to obtain a sugar mixture;
filtering the sugar mixture;
concentrating the filtered sugar mixture; and
precipitating galactose by steps comprising adding an alcohol to the concentrated sugar mixture.

2. The method of claim 1, wherein the red algae is one or more selected from the group consisting of the genus Chondrus, Eucheuma, Gigartina, Pterocladia, Hypnea, Iridaea, Kappaphycus, Gellidium, and Gracilaria.

3. The method of claim 1, wherein the saccharification is performed at a temperature of about 80 to 150° C.

4. The method of claim 1, wherein the saccharification is performed by hydrolysis of the red algae.

5. The method of claim 4, wherein the hydrolysis is performed by steps comprising adding an acid at a concentration of about 0.1% (w/v) to 15% (w/v).

6. The method of claim 5, wherein the acid is one or more selected from the group consisting of sulfuric acid (H₂SO₄), hydrochloric acid (HCl), bromic acid (HBr), nitric acid (HNO₃), acetic acid (CH₃COOH), formic acid (HCOOH), perchloric acid (HClO₄), phosphoric acid (H₃PO₄), and para toluenesulfonic acid (PTSA).

7. The method of claim 1, wherein the filtration of red algae is performed by silica gel chromatography or filtration using a filter.

8. The method of claim 1, wherein the agarase is obtained from Saccharophagus degradans 2-40.

9. The method of claim 1, wherein the agarase-containing solution is obtained by steps comprising culturing Saccharophagus degradans 2-40; removing Saccharophagus degradans 2-40 from a culture medium; and concentrating an agarase remaining in the culture medium.

10. The method of claim 9, wherein Saccharophagus degradans is cultured at a temperature of about 30 to 40° C. for about 36 to 72 hours.

11. The method of claim 1, wherein the sugar mixture is filtered by steps comprising filtering the sugar mixture using a column chromatography, and additionally filtering the sugar mixture using a microfilter.

12. The method of claim 11, wherein the column chromatography contains a silica gel having an average particle diameter of about 0.1 to 0.5 mm.

13. The method of claim 11, wherein the microfilter has a pore size of about 0.45 to 0.9 μm.

14. The method of claim 1, wherein the filtering the sugar mixture is performed at a flow rate of about 0.1 to 100 mL/min.

15. The method of claim 1, wherein the concentrating is performed by distillation of the filtered sugar mixture under vacuum.

16. The method of claim 1, wherein the concentrating is performed at a temperature of about 30 to 60° C.

17. The method of claim 1, wherein the concentrating is performed at a pressure of about 10 to 120 mbar.

18. The method of claim 1, wherein the precipitating is performed at a temperature of about −10° C. to 25° C.

19. The method of claim 1, wherein the alcohol added in the precipitating is one or more selected from the group consisting of methanol, ethanol, and propanol.

20. The method of claim 1, further comprising additional filtering after the precipitating to obtain galactose particles.
